(19) United States
(12) Patent Application Publication
(10) Pub. No.: US 2009/0053245 A1
(43) Pub. Date: Feb. 26, 2009

(54) Pharmacokinetics and Efficacy of Anti-Angiogenic Drugs and Drugs Treating Diseases of the Blood

(76) Inventors: Mitchell W. Mutz, La Jolla, CA (US); Andre L. Marquis, San Carlos, CA (US)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(21) Appl. No.: 12/129,487

(22) Filed: May 29, 2008

Related U.S. Application Data
(60) Provisional application No. 60/932,359, filed on May 29, 2007.

(51) Int. Cl. A61K 39/395; A61K 31/445; A61P 31/00

(52) U.S. Cl. 424/174.1; 514/326

(57) ABSTRACT

A method for modulating at least one pharmacokinetic property of an anti-angiogenic or blood disease or steroid therapeutic and efficacy upon administration to a host is provided. One administers to the host an effective amount of a bifunctional compound of less than about 5000 Daltons comprising the anti-angiogenic or blood disease or steroid therapeutic or an active derivative thereof and a pharmacokinetic modulating moiety. The pharmacokinetic modulating moiety binds to at least one intracellular protein. The bifunctional compound has at least one modulated pharmacokinetic property upon administration to the host as compared to a free drug control that comprises the anticancer therapeutic as well as enhanced efficacy not due to compound degradation. It is preferred that the pharmacokinetic modulating moiety has a mass of less than 1100 Daltons.

### CROSS-REFERENCE TO RELATED APPLICATIONS

This application claims priority to U.S. Provisional Application Ser. No. 60/932,359, filed on May 29, 2007, which is incorporated by reference in its entirety.

### TECHNICAL FIELD

This invention relates generally to pharmacology and more specifically to the modification of known active agents to give them more desirable properties.

### BACKGROUND

Diseases of the blood have been an important area of clinical research for many years. Recently, targeting therapeutics to blood cells as well as targeting drugs to angiogenic proteins that help form blood vessels has allowed progress in treating cancer, chronic obstructive pulmonary disorder, and cystic fibrosis. For example, clinicians have used direct erythrocyte loading to enhance compound half-life, as well as reduce toxicity, and as a means to target a therapeutic more effectively (M Magnani, L Rossi, A Fraternale, M Bianchi, A Antonelli, R Crinelli and L Chiarantini, Gene Therapy (2002) 9, 749-751). The method disclosed by Magnani et al. requires the patient to donate blood for therapeutic loading, and then a second visit to re-introduce the treated blood cells. Molecularly targeting compounds into erythrocytes for drug delivery would spare the patient from the blood withdrawal and re-introduction currently required.

An example of progress in treating a blood disease has been Imanitib, used mainly for chronic myelogenous leukemia (CML). Imanitib's long half-life is closely associated with a high degree of blood-protein binding and has represented a true breakthrough in the treatment of CML. More recently, compounds showing a high degree of intracellular retention in whole blood have exhibited good efficacy (vide infra) and have promise as improved pharmaceutical entities.

Paclitaxel has also been one of the most important chemotherapeutic agents used in the last ten years. Although effective in many applications such as ovarian cancer and metastatic breast cancer, paclitaxel does produce toxic side effects like all chemotherapeutic agents in use today. These side effects range from neutropenia to mucosititis. Patient compliance and drug toxicity have always been major issues with chemotherapeutics due to the frequency of dosage and co-administration of other cancer therapeutics. A reduction in dosage frequency would represent an improvement in quality of life for the patient and a lower chance of toxic side effects due to decreased production of secondary metabolites, especially in the liver. Paclitaxel also does not exhibit optimal retention in blood cells and therefore has not been used to treat liquid tumors. Optimizing the retention of paclitaxel in target cells can open new indications for the treatment of liquid tumors.

Another technical issue for blood cell loading of paclitaxel is caused by hepatic metabolism of paclitaxel and tendency to produce polar species of the therapeutic agent that are no longer are capable of crossing cell membranes. For example, the 6-alpha- and 3′-p-hydroxy metabolites of paclitaxel have a lower ability to cross cell membranes relative to the paclitaxel parent compound. Since many chemotherapeutic agents affect chromosomes and other intracellular components, the polar secondary metabolites are generally less effective than the parent compound. Although recent cancer chemotherapeutics require a reduced dosage burden compared with earlier drugs, there is still significant opportunity for improving chemotherapeutics by reducing first pass clearance via cytochrome P450 enzymes and increasing the drug half-life in the circulation. In addition, sustaining the ability of the drug to cross the cell membrane and remain inside cells can also prolong the circulating half-life by avoiding extra-cellular enzymes that degrade the parent compound. Moreover, where the mode of drug activity is intracellular, a reduced resident time in the extracellular space is desirable.

Examples of cancer chemotherapeutics that have diminished half-lives due to poor pharmacokinetics are methotrexate, vinblastine, paclitaxel and vincristine, among others. Another effect of diminished half-life is that many chemotherapeutics are particularly effective during certain parts of the cell cycle (mitosis, meiosis, cell division, etc.) and if the drug has rapid clearance, it will not be present at the relevant part of the cell cycle. Other drugs with suboptimal half-lives include dexamethasone, a corticosteroid.

Previous methods to improve pharmacokinetics (PK) of cancer chemotherapeutics include: medicinal chemistry-based analog synthesis, pro-drug strategies, improved formulation, and co-administration with P450 and P-glycoprotein inhibitors. Additionally, drugs such as methotrexate and paclitaxel have been covalently bound to albumin. While this method has yielded improvements in pk (increased persistence in the circulation relative to the parent compound), the high cost of preparing drug directly conjugated to protein is a disadvantage of the direct conjugation method. Moreover, direct protein drug conjugates are not amenable to oral delivery due to the low pH in the gut and digestive enzymes. Regardless of the methods employed, these approaches share a desired outcome: improve pharmacokinetics to make treatment easier for patients. However, these methods have not yielded an agent that requires a substantially lower dose (e.g., once or twice per week or month) over the prior chemotherapeutic agents and are relatively non-toxic compared with prior chemotherapeutics. Low toxicity is an especially important consideration for metronomic chemotherapy, where a patient may receive a low drug dose for a year or more to prevent the recurrence of a cancer that is not detectable by conventional medical imaging.

An additional benefit to the patient of a bifunctional approach is the simultaneous optimization of both pharmacokinetics and efficacy not due to pharmacokinetics This presents a technical challenge since properties such as enhanced steric bulk may shield a drug from enzymatic degradation, but steric bulk may prevent a drug from interacting with the active site of a target compound. In such a case, the enhanced benefit of the improved pk may be offset by the decreased affinity of the bifunctional compound relative to the parent (monofunctional) compound. In addition to affinity, other drug properties that may be optimized by the bifunctional approach are binding kinetics of the drug moiety to the drug target and binding of the pharmacokinetic modulating moiety, abbreviated PMM, to the PMM's target, typically an endogenous protein.

There is therefore still a need in the art for chemotherapeutics and associated dosage forms that have reduced first pass clearance and/or improved pharmacokinetics, while being relatively economical to produce, and are still effective in maintaining affinity for the drug target. Ideally, these drugs associate with a non-target protein to take advantage of steric bulk, but the non-covalent association must also allow the drugs to bind effectively to the target as well.

### SUMMARY OF THE INVENTION

In an embodiment of this invention, a method for modulating at least one pharmacokinetic property of a therapeutic upon administration to a host is provided and simultaneously enhancing efficacy with a non-pharmacokinetic property such as binding constant or k_{off} rate constant. The therapeutic target is a disease of the blood or an angiogenic protein such as VEGF, bFGF, PDGF. Examples of blood diseases include sickle cell anemia, malaria and leukemia Moreover, the therapeutic gains efficacy and improved pk by sequestration in the intracellular space. The sequestration lowers the toxicity of the therapeutic and also provides for an extended controlled release of the therapeutic when the drug target is an angiogenic protein or target related to a blood disease such as malaria, sickle cell anemia, or leukemia. The sequestration also finds use where the therapeutic is a steroid and whose controlled release may be realized by sequestration in the intracellular space of blood cells. Erythrocyte loading of dexamethasone has been shown to increase the efficacy of this steroid in the therapy of cystic fibrosis as described by Magnani, et al. as referenced above.

In an aspect of the invention, one administers to the host an effective amount of a bifunctional compound of less than about 5000 Daltons comprising the therapeutic or an active derivative thereof and a pharmacokinetic modulating moiety. The pharmacokinetic modulating moiety binds to at least one intracellular protein. The bifunctional compound has at least one modulated pharmacokinetic property and enhanced efficacy not due to pharmacokinetic optimization upon administration to the host as compared to a free drug control that comprises the anticancer therapeutic.

In a further embodiment of this invention, a bifunctional compound comprising a cancer chemotherapeutic functionality and a pharmacokinetic modulating moiety are provided.

In a further aspect of the invention, a bifunctional compound is provided in a pharmaceutical formulation that sustains the ability of the compound to cross cell membranes and avoid catalysis by cytochrome p450 enzymes and other drug-degrading catalysts inside cells.

In a further aspect of the invention, biasing the drug to remain inside cells increases efficacy by a two-fold mechanism: avoiding extracellular and intracellular Cytp450 enzymes and avoiding intracellular degradation by enzymes via an association with a non-target intracellular protein which confers protection from intracellular enzymes. The non-target protein must still allow binding to the drug target and optimally enhances the binding affinity measured directly by the association constant, Ka, or indirectly by IC₅₀ measurements. The intracellular bias is created using ligands for intracellular proteins.

In a further aspect, the bifunctional drug has lower toxicity than the parent compound because a lower dose is required to achieve equivalent efficacy due to enhanced concentration/hour (area under the curve) and that non-target binding is directed to a high abundance, non-target protein (albumin, HSP90, FKBP12, etc.)

In a further aspect of the invention, the bifunctional drug is particularly effective in reducing the size of drug resistant tumors since the enhanced binding to the non-target protein has a lower equilibrium dissociation constant or dissociation rate constant than the dissociation constant or dissociation rate constant of the monofunctional compound with protein complexes that pump drugs and other xenobiotics out of cells.

A further embodiment of the invention is targeting the drug to diseased blood cells to enhance the drug efficacy.

A further aspect of the invention is to enhance the efficacy of drugs which are anti-angiogenic.

A further aspect of the invention is that the linker length has been selected to allow binding of the bifunctional to the drug moiety and the PMM binding target at the same time.

A further aspect of the invention is using a bifunctional to target cancer stem cells and drug resistant tumors.

### FIGURES

FIG. 1 depicts the structure of SLF (synthetic ligand for FKBP) linked to a modular linker and target binding moiety, for example an anticancer therapeutic. Due to the modular nature of the synthesis, the linker group and target-binding group may be readily altered.

FIG. 2 illustrates how the steric bulk of a protein can confer protection from enzymes.

In FIG. 3, the left side depicts the bimodal binding character of FK506 whereby it binds both FKBP and calcineurin. The schematic on the right depicts how the calcineurin-binding mode can be eliminated by substituting a linker and target binding moiety. In this manner, FK506 can simultaneously target FKBP and bind a second protein. Synthetic ligands with no affinity for calcineurin such as SLF may also be used.

In FIG. 4A, we see the structure of FK506 bound to curcumin. FIG. 4B illustrates how FK506-curcumin is protected from CYP3a4, a P450 enzyme, in the presence of FKBP. FIG. 4C gives a schematic of the Invitrogen assay used.

In FIG. 5, the left side illustrates sample linkers that could be employed in a modular synthetic scheme.

FIG. 6 exhibits a synthetic scheme for a bifunctional form of paclitaxel. See S. Wang et al., Bioorg. Med. Chem. Lett., 16, 2628-2631 (2006).

FIG. 7 illustrates the efficacy of a bifunctional paclitaxel drug in cell culture. It provides an example of enhanced efficacy of bifunctional paclitaxel in the absence of drug-degrading enzymes. The lower o.d. indicates more tumor cell growth inhibition by paclitaxel-SLF (right bar in each pair).

FIG. 8 shows the difference in partitioning between extra- and intracellular space due to the presence of the recruiter ligand moiety in an in vivo mouse model study.

FIG. 9 shows the effect of area under the curve for a bifunctional compound in mice vs. a monofunctional compound. Compound was administered via a tail vein injection to mimic intravenous drug administration. The data shows a 25 fold increase in area under the curve for the bifunctional vs. the monofunctional.

FIG. 10 shows the efficacy of the paclitaxel bifunctional in a xenograft tumor mouse model vs. a vehicle control containing the Cremaphor-ethanol solvent only.

FIG. 11 shows the partitioning of a protease inhibitor bifunctional between blood cells and plasma as determined in an in vivo study in mice.

FIG. 12 depicts a synthetic methodology for an acenocoumarin-SLF bifunctional compound.

FIG. 13 depicts the coagulation time as a function of time after oral administration for a warfarin-SLF bifunctional compound, warfarin, and a vehicle control.

FIG. 14 depicts the intracellular sequestration achieved by means of a 4-methoxy amprenavir-SLF bifunctional molecule as compared to 4-methoxy amprenavir itself.

FIG. 15 depicts the increase activity achieved by means of a 4-methoxy amprenavir-SLF bifunctional molecule as compared to 4-methoxy amprenavir itself.

### DETAILED DESCRIPTION OF PREFERRED EMBODIMENTS

Before describing the present invention in detail, it is to be understood that this invention is not limited to specific solvents, materials, or device structures, as such may vary. It is also to be understood that the terminology used herein is for the purpose of describing particular embodiments only, and is not intended to be limiting.

As used in this specification and the appended claims, the singular forms “a,” “an,” and “the” include both singular and plural referents unless the context clearly dictates otherwise. Thus, for example, reference to “an active ingredient” includes a plurality of active ingredients as well as a single active ingredient, reference to “a temperature” includes a plurality of temperatures as well as single temperature, and the like.

The term “bifunctional compound” refers to a non-naturally occurring compound that includes a pharmacokinetic modulating moiety and a drug moiety, where these two components may be covalently bonded to each other either directly or through a linking group. The term “drug” refers to any active agent that affects any biological process. Bifunctional compounds may have more than two functionalities.

The pharmacokinetic modulating moiety may be a peptide or protein and may also be an enzyme or nucleic acid. Similarly, the drug moiety may also be peptide, protein, enzyme, or nucleic acid.

Active agents which are considered drugs for purposes of this application are agents that exhibit a pharmacological activity. Examples of drugs include active agents that are used in the prevention, diagnosis, alleviation, treatment or cure of a disease condition.

By “pharmacologic activity” is meant an activity that modulates or alters a biological process so as to result in a phenotypic change, e.g. cell death, cell proliferation etc.

By “pharmacokinetic property” is meant a parameter that describes the disposition of an active agent in an organism or host. Representative pharmacokinetic properties include: drug half-life, hepatic first-pass metabolism, volume of distribution, degree of blood serum protein, e.g. albumin, binding, etc, degree of tissue targeting, cell type targeting.

By “half-life” is meant the time for one-half of an administered drug to be eliminated through biological processes, e.g. metabolism, excretion, etc.

By “hepatic first-pass metabolism” is meant the propensity of a drug to be metabolized upon first contact with the liver, i.e. during its first pass through the liver.

By “volume of distribution” is meant the distribution and degree of retention of a drug throughout the various compartments of an organism, e.g. intracellular and extracellular spaces, tissues and organs, etc.

The term “efficacy” refers to the effectiveness of a particular active agent for its intended purpose, i.e. the ability of a given active agent to cause its desired pharmacologic effect.

The term “host” refers to any mammal or mammalian cell culture or any bacterial culture.

Where the term cancer is used, it is understood that the invention may be employed on relative chemotherapeutics such as found in other any type of cancer including those cancers found in non-human species or human variants.

Where the term “intracellular” protein is used, this includes any protein that resides predominantly in the intracellular space but may optionally reside as a transmembrane or receptor protein.

The term “metronomic therapy” refers to long-term preventive anti-cancer chemotherapy where a drug is administered over a much longer term (many months instead of weeks) to avoid recurrence of tumors. Normally, toxicity dictates the use of chemotherapy at very low doses that compromise the effectiveness of this mode of therapy.

The term “biomoiety” refers to a protein, DNA, RNA, ligand, carbohydrate, lipid, or any other component molecule of a prokaryotic or eukaryotic organism.

The term “liquid tumor” refers to any variety of leukemia or cancer which substantially affects blood cells such as red blood cells, white blood cells, macrophages, T cells, B-cells, or other circulating tumor cell including cancer stem cells.

The term “non-pharmacokinetic properties” may include binding constants, off rate or on rate constants of the drug moiety and PMM for their targets. Additional properties may include drug solubility, formulation, and permeability across membranes.

Where FK506 is used, variants or analogs of FK506 are included, such as rapamycin, pimecrolimus, or synthetic ligands of FK506 binding proteins (SLFs) such as those disclosed in U.S. Pat. Nos. 5,665,774, 5,622,970, 5,516,797, 5,614,547, and 5,403,833 or described by Holt et al., “Structure-Activity Studies of Synthetic FKBP Ligands as Peptidyl-Prolyl Isomerase Inhibitors,” Bioorganic and Medicinal Chemistry Letters, 4(2):315-320 (1994).

In an embodiment of this invention, a method for modulating at least one pharmacokinetic property of an anticancer therapeutic upon administration to a host is provided. One administers to the host an effective amount of a bifunctional compound of less than about 5000 Daltons comprising the anticancer therapeutic or an active derivative thereof and a pharmacokinetic modulating moiety. The pharmacokinetic modulating moiety binds to at least one intracellular protein. The bifunctional compound has at least one modulated pharmacokinetic property upon administration to the host as compared to a free drug control that comprises the anticancer therapeutic.

Bifunctional compound in general have aroused considerable interest in recent years. See, for example, U.S. Pat. Nos. 6,270,957, U.S. Pat. No. 6,316,405, U.S. Pat. No. 6,372,712, U.S. Pat. No. 6,887,842, and U.S. Pat. No. 6,921,531. ConjuChem (Montreal, Canada) scientists have shown that covalent coupling of insulin to human serum albumin can improve the half-life from 8 hours to over 48 hours. Xenoport (Santa Clara, Calif.) has pioneered attachment of receptor ligands to improve drug uptake and distribution. Human trials of methotrexate-albumin conjugates revealed that the modified methotrexate had half-lives of up to two weeks compared with 6 hours for unmodified methotrexate. Other examples include PEGylation of growth factors and attachment of folate groups that “target” anti-cancer drugs. All these strategies use modification of a “parent” drug to provide new binding profiles or enhanced protection from degradation.

More recently, a team including one of the inventors attached SLF to ligands for amyloid beta. Amyloid beta oligomers are believed to underlie the neuropathology of Alzheimer's disease. Therefore, methods to decrease amyloid aggregation are of therapeutic interest. Amyloid ligands, such as congo red or curcumin (above), can be synthetically coupled to FK506 or SLF. The resulting bifunctional compound binds both FKBP and amyloid beta. These molecules are potent inhibitors of amyloid aggregation and they block neurotoxicity in cell culture. See Jason E. Gestwicki et al., “Harnessing Chaperones to Generate Small-Molecule Inhibitors of Amyloid β Aggregation,” Science 306:865-69 (2004).

Bifunctional compounds of the type employed in the present invention are generally described by the formula:

X-L-Z

wherein:

X is a drug moiety;

L is a bond or linking group; and

Z is a pharmacokinetic modulating moiety,

with the proviso that X and Z are different. Thus, as may be seen, a bifunctional compound is a non-naturally occurring or synthetic compound that is a conjugate of a drug or derivative thereof and a pharmacokinetic modulating moiety, where these two moieties are optionally joined by a linking group.

In bifunctional compounds used in the invention the pharmacokinetic modulating and drug moieties may be different, such that the bifunctional compound may be viewed as a heterodimeric compound produced by the joining of two different moieties. In many embodiments, the pharmacokinetic modulating moiety and the drug moiety are chosen such that the corresponding drug target and any binding partner of the pharmacokinetic modulating moiety, e.g., a pharmacokinetic modulating protein to which the pharmacokinetic modulating moiety binds, do not naturally associate with each other to produce a biological effect.

The bifunctional compounds are typically small. As such, the molecular weight of the bifunctional compound is generally at least about 100 D, usually at least about 400 D and more usually at least about 500 D. The molecular weight may be less than about 800 D, about 1000 D, about 1200 D, or about 1500 D, and may be as great as 2000 D or greater, but usually does not exceed about 5000 D. The preference for small molecules is based in part on the desire to facilitate oral administration of the bifunctional compound. Molecules that are orally administrable tend to be small.

The pharmacokinetic modulating moiety modulates a pharmacokinetic property, e.g. half-life, hepatic first-pass metabolism, volume of distribution, degree of albumin binding, etc., upon administration to a host as compared to free drug control. By modulated pharmacokinetic property is meant that the bifunctional compound exhibits a change with respect to at least one pharmacokinetic property as compared to a free drug control. For example, a bifunctional compound of the subject invention may exhibit a modulated, e.g. longer, half-life than its corresponding free drug control. Similarly, a bifunctional compound may exhibit a reduced propensity to be eliminated or metabolized upon its first pass through the liver as compared to a free drug control. Likewise, a given bifunctional compound may exhibit a different volume of distribution that its corresponding free drug control, e.g. a higher amount of the bifunctional compound may be found in the intracellular space as compared to a corresponding free drug control. Analogously, a given bifunctional compound may exhibit a modulated degree of albumin binding such that the drug moiety's activity is not as reduced, if at all, upon binding to albumin as compared to its corresponding free drug control. In evaluating whether a given bifunctional compound has at least one modulated pharmacokinetic property, as described above, the pharmacokinetic parameter of interest is typically assessed at a time at least 1 week, usually at least 3 days and more usually at least 1 day following administration, but preferably within about 6 hours and more preferably within about 1 hour following administration.

The linker L, if not simply a bond, may be any of a variety of moieties chosen so that they do not have an adverse effect on the desired operation of the two functionalities of the molecule and also chosen to have an appropriate length and flexibility. The linker may, for example, have the form F₁—(CH₂)ₙ—F₂ where F₁ and F₂ are suitable functionalities. A linker of this sort comprising an alkylene group of sufficient length may allow, for example, for the free rotation of the drug moiety even when the pharmacokinetic modulating moiety is bound. Alternatively, a stiffer linker with less free rotation may be desired. The hydrophobicity or hydrophobicity of the linker is also a relevant consideration. FIG. 5 depicts some precursors which may be used for the linker (with the carboxyl functionality protected).

The drug moiety X may, in certain embodiments of the invention, preferably be an anticancer therapeutic. The drug moiety may be derived from a known anticancer therapeutic, which is preferably effective against one or more types of cancer. The drug moiety preferably has a functionality which may readily and controllably be made to react with a linker precursor. The known chemotherapeutics are generally susceptible to metabolism and subsequent deactivation by hepatic first-pass or subsequent pass clearance mechanisms. Cancer chemotherapy is an active area of research.

Increased targeting to the blood caused by the pharmacokinetic modulating moiety may result in superior efficacy for anticancer agents. To begin with, a growing tumor tends to become well perfused. Certain anticancer agents may directly target angiogenesis in the tumor. Others may target cancers such as liquid tumors which affect the circulation. Such anticancer agents would be expected to benefit from increased targeting to the blood.

Certain of the concepts of this invention have applicability to other drug moieties besides cancer chemotherapeutics. In general, bifunctional compounds may usefully be made with any drug having a suitable moiety capable of reacting with linkers and which has a need for pharmacokinetic modulation. Thus, for example, drugs having a strong first-pass effect may be candidates for incorporation into a bifunctional compound.

Bifunctional compounds may be made, for example, with antimalarial drugs such as chloroquine, mefloquine, quinidine gluconate, pyrimethaminesulfadoxine, pyrimethamine-sulfadiazine, mefloquine, artesunate, atovaquone, and proguanil. An advantage of this is that increased targeting to the blood caused by the pharmacokinetic modulating moiety may result in superior efficacy for such a bifunctional compound compared to the base drug.

Increased targeting to the intracellular components of blood caused by the pharmacokinetic modulating moiety may also result in superior efficacy for steroidal anti-inflammatory agents. Examples of these drugs are cortisol, cortisone, fludrocortisone, prednisone, prednisolone, 6α-methylprednisolone, triamcinolone, betamethasone, and dexamethasone.

Increased targeting to the intracellular space of blood cells may also result in superior efficacy for anti-angiogenic agents. Examples of these drugs are bortezomib, thalidomide, bevacizumab, erlotinib, pegaptanib, endostatin, sorafenib, lenalidomide, sunitinib, and ranibizumab.

FIG. 14 shows the increased targeting to intracellular space which has been achieved with a 4-methoxy amprenavir-SLF conjugate. FIG. 15 shows the increased activity of this conjugate which is believed to be related to its increased targeting to intracellular space.

In general, the pharmacokinetic modulating moiety Z will be one which is capable of reversible attachment to a common protein, meaning one which is abundant in the body or in particular compartments of the body or particular tissue types. Common proteins include, for example, FK506 binding proteins, cyclophilin, tubulin, actin, heat shock proteins, and albumin. Common proteins are present in concentrations of at least 1 micromolar, preferably at least 10 micromolar, more preferably at least 100 micromolar, and even more preferably 1 millimolar in the body or in particular compartments or tissue types. The pharmacokinetic modulating moiety should, like the drug, have a moiety which is capable of reacting with suitable linkers.

It is desirable for at least some embodiments of the present invention that the binding of the pharmacokinetic modulating moiety Z to a common protein be such as to sterically hinder the activity of common metabolic enzymes such as CYP450 enzymes when the bifunctional compound is so bound. Persons of skill in the art will recognize that the effectiveness of this steric hindrance depends, among other factors, on the conformation of the common protein in the vicinity of the pharmacokinetic modulating moiety's binding site on the protein, as well as on the size and flexibility of the linker. The choice of a suitable linker and pharmacokinetic modulating moiety may be made empirically or it may be made by means of molecular modeling of some sort if an adequate model of the interaction of candidate pharmacokinetic modulating moieties with the corresponding common proteins exists. The linker choice must balance parameters of length, hydrophobicity, attachment point to the drug target, and attachment point to the ligand.

The attachment point and linker characteristics are preferably selected based on structural information such that the inhibitory potency of the anticancer therapeutic is preserved, giving the desired superior pharmacokinetic characteristics.

Where the pharmacokinetic modulating moiety operates by binding a protein, it may be referred to as a “presenter protein ligand” and the protein which it binds to may be referred to as a “presenter protein.”

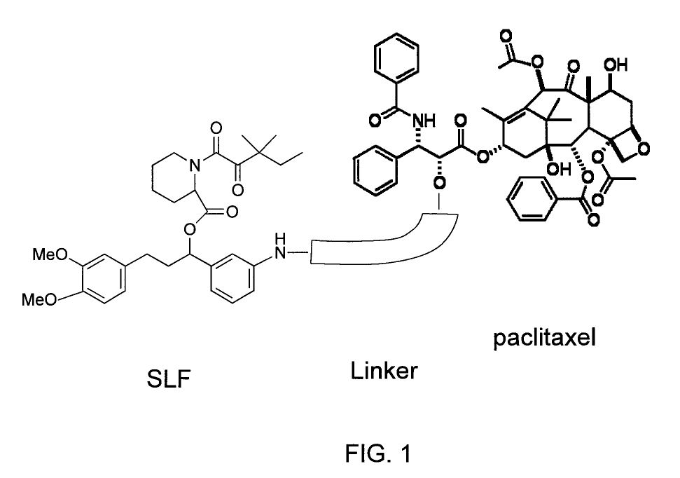

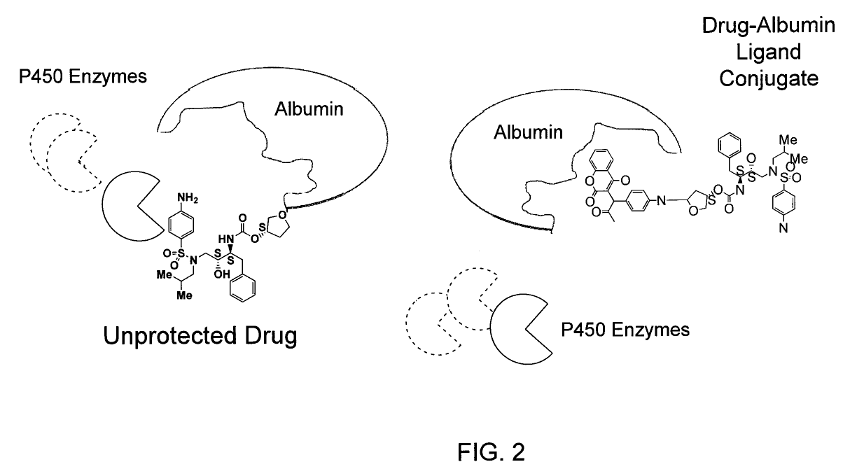

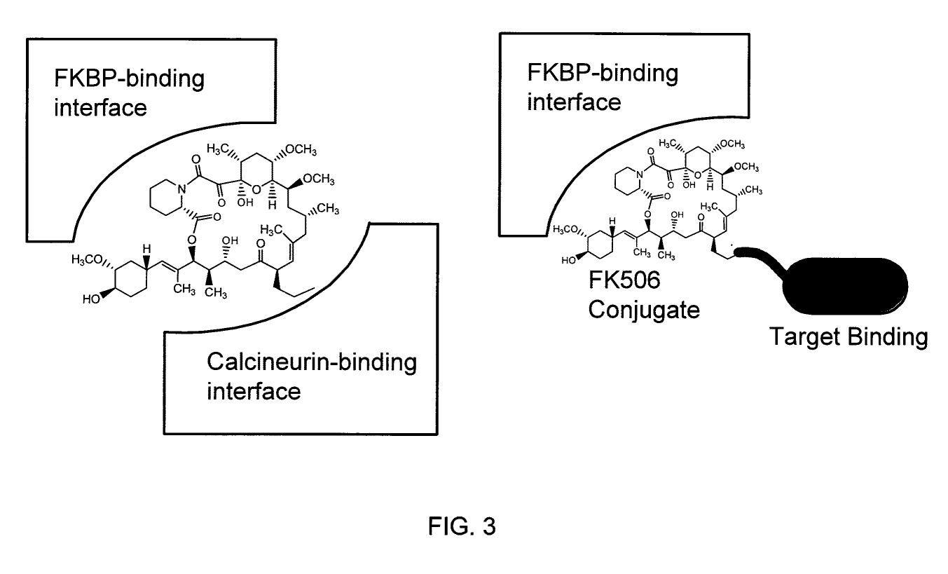

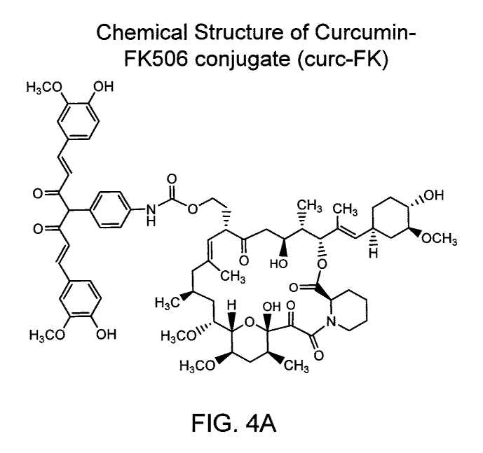

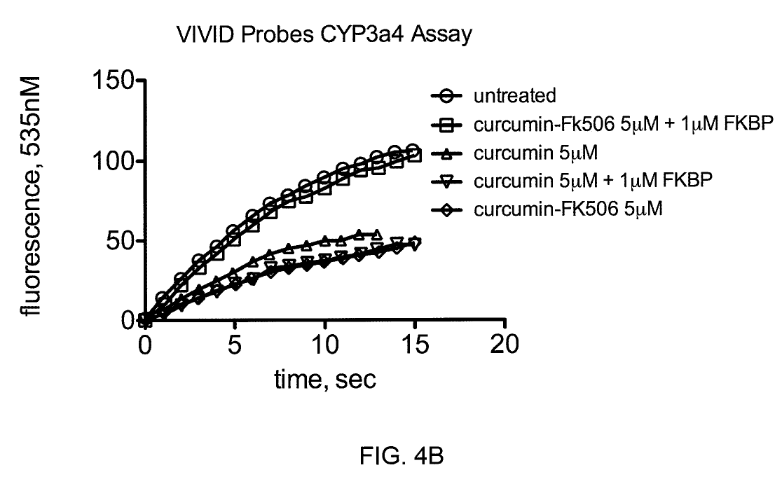

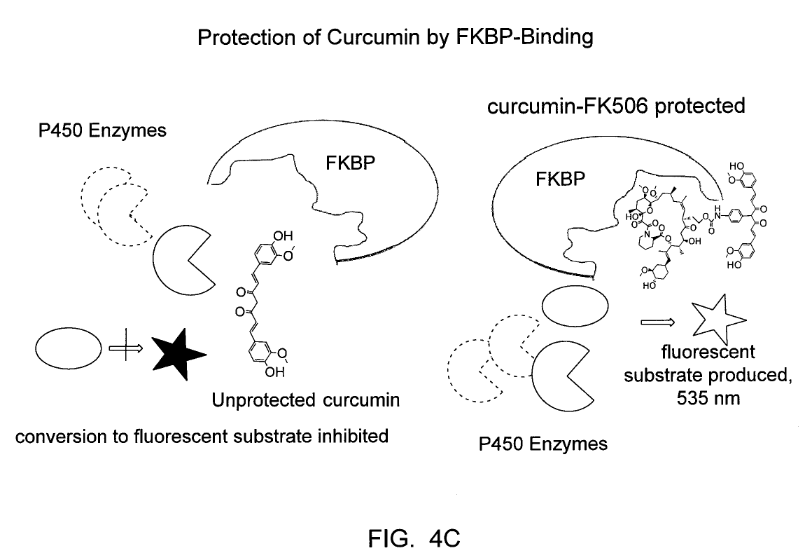

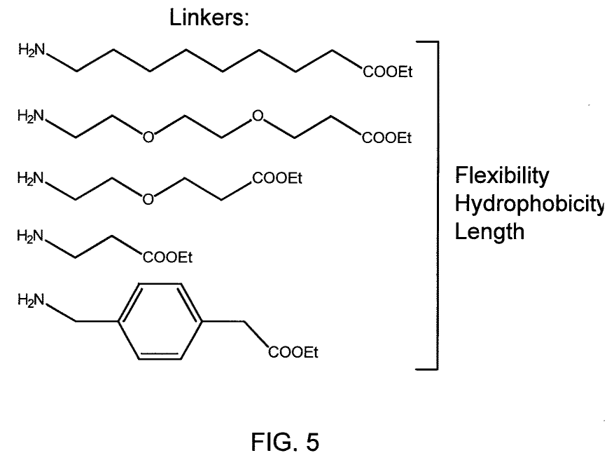

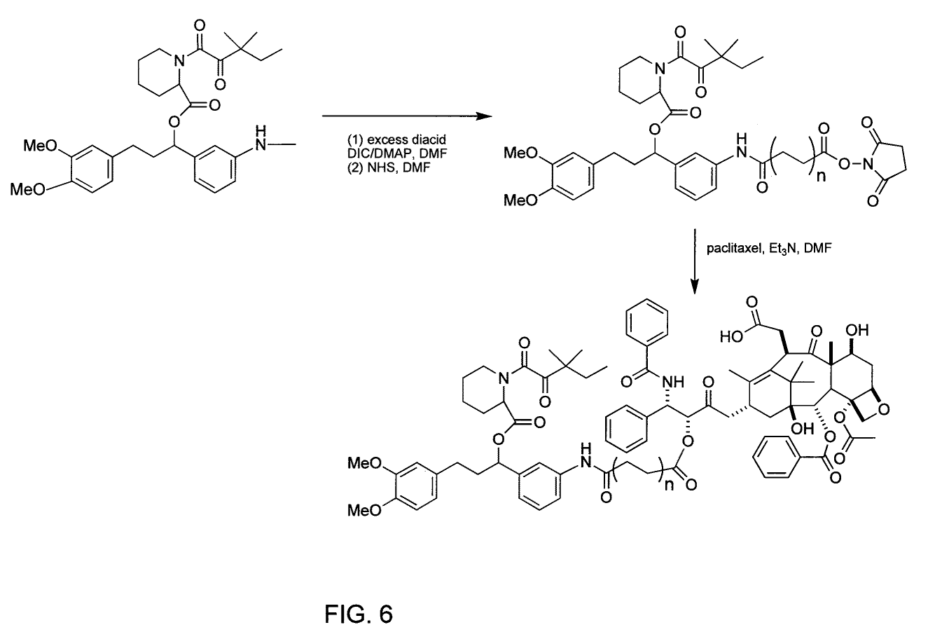

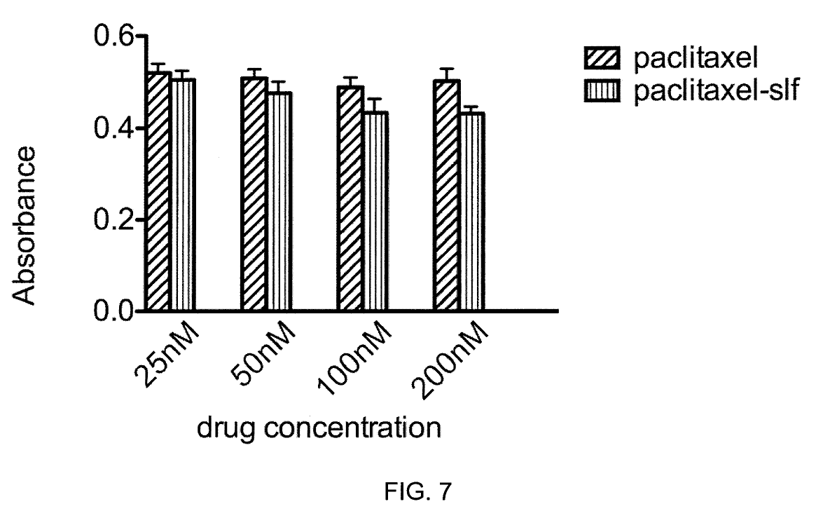

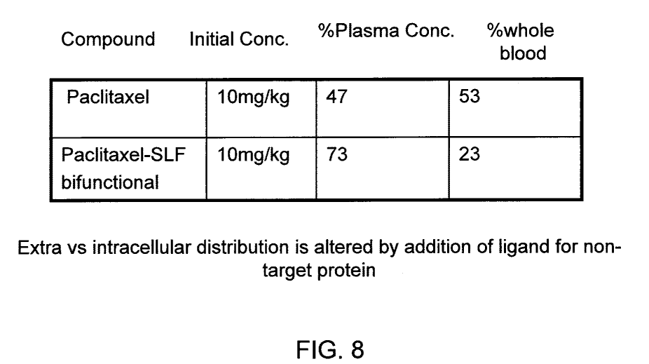

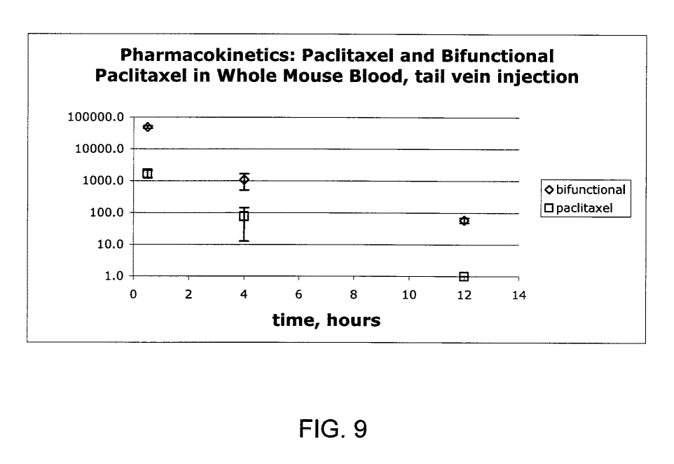

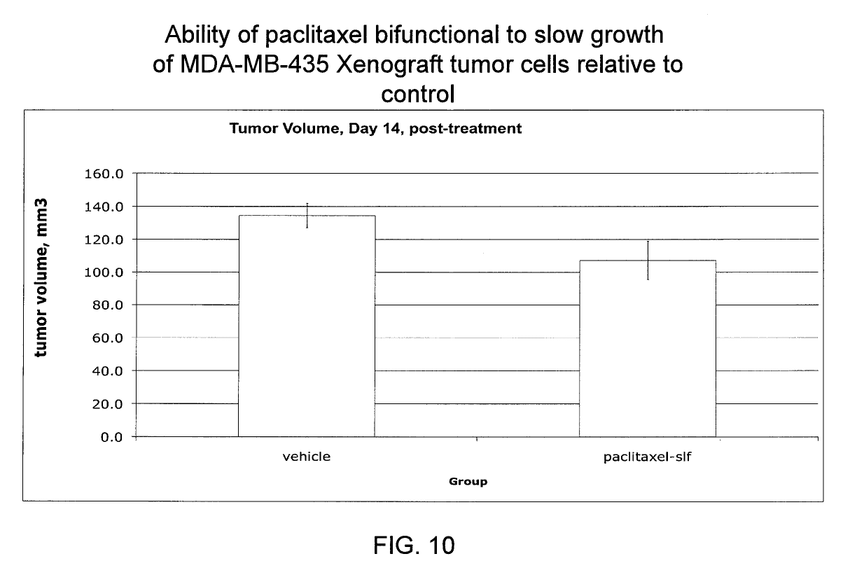

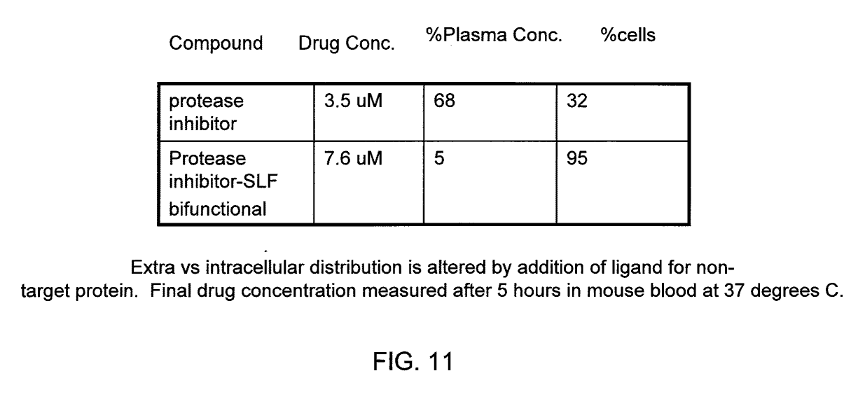

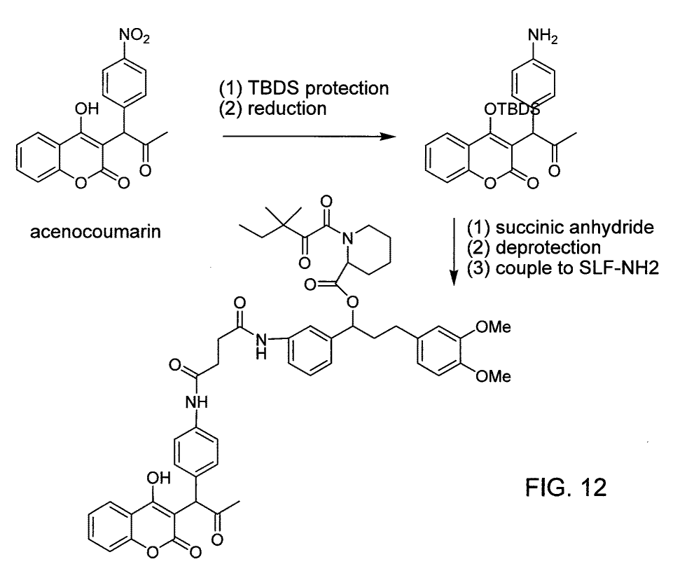

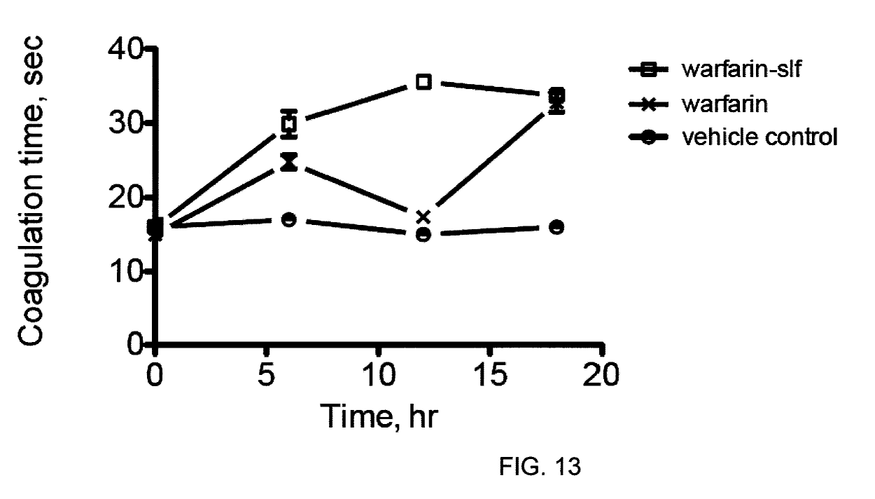

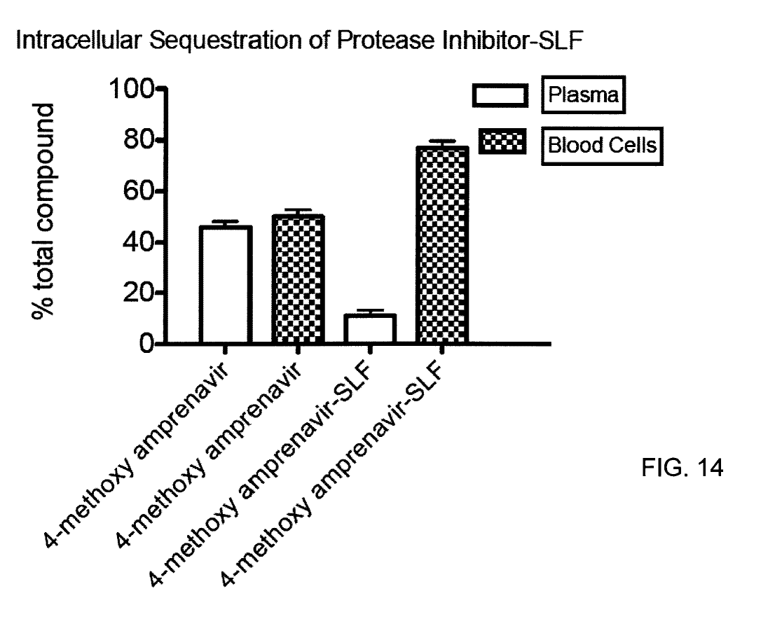

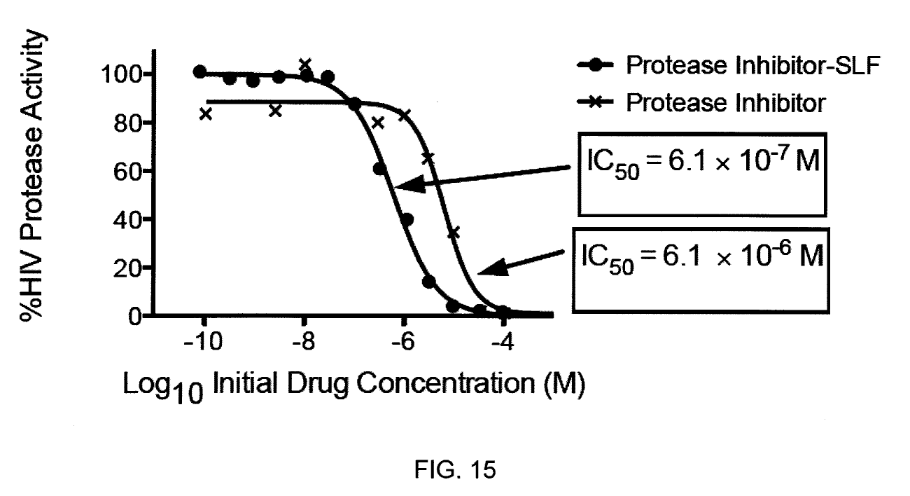

The pharmacokinetic modulating moiety may be, for example, a derivative of FK506, which has high affinity for the FK506-binding protein (FKBP), as depicted for example in FIG. 1. There are many synthetic ligands for FKBP. The abundance of FKBP (millimolar) in blood compartments, such as red blood cells and lymphocytes, makes it likely that a significant proportion of a dose of bifunctional compounds comprising FK506 would partition into blood cells. A mechanism that tends to increase the portion of the chemotherapeutic dose that winds up in red blood cells and CD4+ lymphocytes will have a favorable effect on anti-cancer activity, as these sites are prime targets of chemotherapy. The steric bulk conferred by FKBP would hinder an anticancer therapeutic moiety from fitting into the binding pocket of intracellular enzymes (aldolases, hydroxylases, etc.) and so would prevent degradation via this class of enzymes.

An inactive form of FK506 may be preferable in some applications to avoid the possibility of side effects due to the possible interaction of the active FK506-FKBP complex with calcineurin. It may be advantageous to use FKBP binding molecules such as synthetic ligands for FKBP (SLFs) described by Holt et al., supra. This class of molecule is lower molecular weight than FK506, and that is generally advantageous for drug delivery and pharmacokinetics. For illustrative purposes, some diagrams will show examples of the use of FK506, though it should be understood that the same strategy can apply to other ligands of peptidyl prolyl isomerases such as the FKBP proteins and that ligands for other presenter proteins may be employed.

The value of FK506 and other FKBP binding moieties as pharmacokinetic modulating moieties of the invention is further supported by the following. FK506 (tacrolimus) is an FDA-approved immunosuppressant. It has been determined that FK506 can be readily modified such that it loses all immunomodulatory activity but retains high affinity for FKBP. FKBP is an abundant chaperone that is particularly prevalent (a millimolar) in red blood cells (rbcs) and lymphocytes. The complex between FK506-FKBP gains affinity for calcineurin and inactivation of calcineurin blocks lymphocyte activation and causes immunosuppression.

This interesting mechanism of action is derived from FK506's chemical structure. FK506 is bifunctional; it has two non-overlapping protein-binding faces. One side binds FKBP, while the other binds calcineurin. This property provides FK506 with remarkable specificity and potency. Moreover, FK506 has a long half-life in non-transplant patients (21 hrs) and excellent pharmacological profile. In part, this is because FK506 is unavailable to metabolic enzymes via its high affinity for FKBP, which favors distribution into protected cellular compartments (72-98% in the blood). It can be expected that suitable bifunctional compounds with an FKBP-binding pharmacokinetic modulating moiety will likewise possess some favorable characteristics of inactive FK506, namely, good pharmacokinetics and blood cell distribution.

In general, the pharmacokinetic modulating moiety will have a molecular weight less than about 2000 D, less than about 1800 D, less than about 1500 D, less than about 1100 D, or less than about 900 D.

It is also possible to co administer the common protein to which the pharmacokinetic modulating moiety binds with the bifunctional compound in order to modify the pharmacokinetics to a greater degree than would be possible with just the native concentration of the common protein.

In a further embodiment of this invention, a method is provided for synthesizing a bifunctional compound comprising anticancer therapeutic functionality and the ability to bind to a common protein.

The synthesis of the bifunctional compound starts with a choice of suitable pharmacokinetic modulating and drug moieties. It is desirable to identify on each of these moieties a suitable attachment point which will not result in a loss of biological function for either one. This is preferably done based on the existing knowledge of what modifications result or do not result in a biological function. On that basis, it may reliably be conjectured that certain attachment points on the pharmacokinetic and drug moieties do not affect biological function. Likewise, in FIG. 6 one sees a secondary amine functions on SLF, which can serve as an attachment point to the boron hydroxyl moiety on bortezomib.

A general synthetic strategy is to locate a secondary amine on the drug moiety at which the drug moiety can be split (so that the secondary amine does not form part of any cycle in the drug moiety). The secondary amine is chosen such that, from experimental or other considerations, it is believed that the drug will retain its efficacy if only the portion of the drug moiety to one side of the secondary amine is present. The portion of the drug moiety to that side of the secondary amine is then synthesized by any appropriate technique, with the secondary amine in the synthesized molecule being protected during synthesis by an appropriate protecting moiety such as Boc. The protecting moiety is then removed, leaving a primary amine which may react with a carboxyl group through a variety of known chemistries for making a peptide bond (see, e.g., J. Mann et al., Natural Products: Their Chemistry and Biological Significance (1994), chapter 3). FIG. 6 gives an example of this synthetic strategy.

In a further aspect of the invention, a bifunctional compound comprising warfarin and a recruiter moiety tending to direct the bifunctional towards intracellular space is provided. The recruiter moiety may be for example an SLF. The warfarin and the recruiter moiety may be joined by a linker. The mechanism of action of warfarin is believed to be by acting upon vitamin K epoxide reductase intracellularly. The bifunctional compound may also comprise an analog, derivative, or fragment of warfarin or of a rerelated coumarin anticoagulant such as acenocoumarol, phenprocoumon, or dicumarol.

In light of the data of Example 8, bifunctional compounds comprising warfarin or related compounds are expected to have a faster onset of action upon oral administration compared to warfarin itself. It is notable that despite the considerably greater mass of the bifunctional compound, a faster onset of action is seen when the same weight of bifunctional and warfarin are administered. This faster action is particularly notable because administering the same weight of bifunctional as warfarin implies administering only approximately one third as many moles of the warfarin active region. The results are also notable because the additional volume of the bifunctional compound does not appear to adversely affect its oral availability. Alternatively, if there is such an adverse effect, it is compensated for by the faster onset of action, believed to be due to greater intracellular targeting.

A quantitative measure of the speed of onset of action is the blood clotting time at a selected time following oral administration. For example, the blood clotting time may be measured at about 8, about 12, about 16, about 24, or about 32 hours following oral administration. (In humans the half-life of warfarin is approximately 32 hours for the more active enantiomer.) For a desirable bifunctional formulation, the blood clotting time at the selected time period following oral administration may be, for example, at least about 125% of the blood clotting time of the base drug at that time, or at least about 150% or at least about 200% of the blood clotting time of the base drug at that time. There are well established standard procedures to measure blood clotting time.

The formulations of this aspect of the invention may be employed in a method of treating a patient having a condition against which the anticoagulant is effective.

In a further aspect of the invention, a bifunctional compound comprising a therapeutic moiety, an optional linker, and an SLF is provided, wherein the oral bioavailability of the therapeutic moiety is not hindered by the SLF.

In a further aspect of the invention, a bifunctional compound comprising an anticancer therapeutic moiety is formulated, for example in the form of a tablet, capsule, parenteral formulation, liposome or nanoliposome to make a pharmaceutical preparation. The pharmaceutical preparation may be employed in a method of treating a patient having cancer against which the anticancer therapeutic moiety is effective. For example, if the anticancer therapeutic moiety is effective against breast cancer, the pharmaceutical preparation may be administered to a patient suffering from breast cancer.

For the preparation of a pharmaceutical formulation containing bifunctional compounds as described in this application, a number of well known techniques may be employed as described for example in Remington: The Science and Practice of Pharmacy, Nineteenth Ed. (Easton, Pa.: Mack Publishing Company, 1995).

In a further aspect of the invention, a bifunctional compound is formulated as part of a controlled release formulation in which an additional controlled release mechanism besides the effect of the pharmacokinetic modulating moiety is employed to achieve desirable release characteristics. The bifunctional compound is as above, comprising a drug moiety, a linker, and a pharmacokinetic modulating moiety. In this aspect of the invention, a drug moiety may be an anticancer therapeutic or a different type of drug.

Drugs which are candidates for bifunctionalization followed by application of other controlled release technologies may belong to a wide variety of therapeutic categories including, but not limited to: analeptic agents; analgesic agents; anesthetic agents; anti-arthritic agents; respiratory drugs, including anti-asthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; anti-inflammatory agents; antimigraine preparations; antinauseants; antiparkinsonism drugs; antipruritics; antipsychotics; antipyretics; antispasmodics; antitubercular agents; anti-ulcer agents; antiviral agents; anxiolytics; appetite suppressants; attention deficit disorder (ADD) and attention deficit hyperactivity disorder (ADHD) drugs; cardiovascular preparations including calcium channel blockers, antianginal agents, central nervous system (CNS) agents, beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine; nutritional agents, such as vitamins, essential amino acids and fatty acids; ophthalmic drugs such as antiglaucoma agents; parasympatholytics; peptide drugs; psychostimulants; sedatives; steroids, including progestogens, estrogens, corticosteroids, androgens and anabolic agents; smoking cessation agents; sympathomimetics; tranquilizers; and vasodilators including general coronary, peripheral and cerebral.

Exemplary drugs presently known to have high first-pass metabolism include HIV chemotherapeutics as discussed above, paclitaxel, methotrexate, vinblastine, verapamil, morphine, lidocaine, acebutolol, isoproterenol, and desipramine. The formation of bifunctional compounds is particularly appropriate for these drugs.

Reference is made to Laurence L. Brunton et al., Goodman & Gilman's The Pharmacological Basis of Therapeutics (11th ed. 2005) for information about drugs which may be candidates for functionalization.

It is to be understood that while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### EXPERIMENTAL

The general method for testing anti-cancer chemotherapeutic-FK506 conjugates is to synthesize the bifunctional compound and test whether the bifunctional version maintains activity against a tumor cell line. The tumor cell line may be grown in cell culture or as part of a xenograft for initial testing. In addition, the P450 susceptibility of the bifunctional compound may be tested in a series of fluorescence-based assays. An important aspect of these experiments was the addition of FKBP sources, such as recombinant protein, red blood cells or lymphocytes. The presence of red blood cells allows the bifunctional to partition into red blood cells and confers protection from extracellular cytochrome P450 metabolism. The desired outcome is prolonged drug lifetime in the presence of an FKBP source combined with potent anti-cancer activity. The synthetic schemes and methods for determining drug lifetime in the presence of P450s will be discussed.

The FDA-drug paclitaxel is a model for generating FK506 or SLF-coupled derivatives of chemotherapeutic agents. Paclitaxel was chosen based on its known defects in pharmacological characteristics.

### Example 1

### FKBP Protection of Curcumin Conjugates

An amyloid ligand, curcumin, is known to be a good substrate for CYP3a4 (a common P450 enzyme). We investigated whether conjugates between curcumin and FK506 would also be substrates for the enzyme. To test this possibility, we utilized a well-known fluorescence-based CYP3a4 assay. This assay, marketed by Invitrogen (Carlsbad, Calif.), under the name VIVID probes, relies on cytochrome-mediated production of a fluorescent marker from a model substrate. When a compound, such as curcumin, binds to the P450, it displaces the substrate and reduces the rate of production of the fluorescent product. When we tested curcumin-FK506 conjugates in this assay, we found that both curcumin and the conjugate were good substrates for the enzyme. Thus, attachment of FK506 did not appear to significantly alter curcumin's susceptibility to degradation by CYP3a4. However, when we supplied a source of human FKBP (in this case, recombinant bacterially-expressed protein), we observed very different results as shown in FIG. 4. In the presence of FKBP, the curcumin-FK506 conjugate is protected from degradation. FKBP was unable to protect unmodified curcumin, which suggests that the ability to bind FKBP is required for FKBP to have a protective effect. It is believed that FKBP protects the curcumin conjugates from degradation by sterically hindering CYP3a4 binding. In the presence of cellular FKBP sources, this effect would be increased because the compartmentalization of the conjugate further reduces availability to P450 enzymes and would sterically hinder intracellular enzymes from degrading the bifunctional compound. Assay results are presented in FIG. 4.

### Example 2

### Synthesis of Conjugate of FK506 and Anti-Angionenic Agent

The linkers shown in FIG. 5 may be coupled to FK506 or SLF via EDC-mediated amide formation followed by deprotection of the newly installed carboxylate. This acid is then used for conjugation to an anti-angiogenic moiety-based molecule as shown in FIG. 6. The linker can be readily altered to enhance solubility or other physical characteristics of the bifunctional compound. The linker must cross cell membranes in the context of the bifunctional molecule. In one preferred embodiment, the linker must permit simultaneous binding of the pharmacokinetic modulating moiety and drug moiety by the bifunctional.

### Examples 3-4

### Synthesis of Paclitaxel-SLF Conjugates

The syntheses of additional paclitaxel-SLF conjugate may proceed in a fashion generally similar to that employed for the FK506-based molecule, as shown in FIG. 6. Linker choice can be important since it can effect compound solubility, transport from the small intestine into the circulation, equilibrium between target and non-target protein binding, efflux via the p-glycoprotein pump, and intra- vs. extracellular distribution.

### Example 5

### Test of Efficacy of Bifunctional Compounds Against a Tumor Cell Line Via IC50 Study

To analyze efficacy of the paclitaxel conjugates of against a tumor cell line, a commercial biochemical assay was used. The MCF7 tumor cell line was grown to confluence in Petri dishes and then either paclitaxel or bifunctional paclitaxel was added to inhibit cell growth as shown in FIG. 7. The cell viability was then measured with the well-known visible indicator, MTT. MTT (3-(4,5-dimethylthiazolyl-2)-2,5-diphenyltetrazolium bromide) is reduced by metabolically active cells, in part by the action of dehydrogenase enzymes, to generate reducing equivalents such as NADH and NADPH. The resulting intracellular purple formazan can be solubilized and quantified by visible spectroscopy. The lower the absorption, the more toxic the compound in this assay.

Similarly, a commercial Invitrogen P2856 assay was used to test for degradation via CYP3a4 in accordance with the manufacturer's directions in the presence of red blood cells.

In the presence of 1 μM FKBP, the bifunctional curcumin moiety is completely protected from degradation via the CYP3a4 as shown in FIG. 4. The monofunctional paclitaxel compound is >70% degraded under the same condition. In the absence of FKBP, the bifunctional is over 70% degraded by the CYP450 enzyme. This cell-free demonstration suggests that if drugs are sequestered inside cells, they will similarly be protected from extra-cellular CYP450. Data illustrating this is given in FIG. 9. The area under the curve for the bifunctional is over 25 times the area under the curve for the monofunctional in data obtained from tail vein injections of mice when measured in whole blood. The area under the curve for the bifunctional is over 60 times the area under the curve for the monofunctional in data obtained from tail vein injections of mice when measured in plasma (data not shown on this curve).

Additionally, FIG. 10 illustrates the in vivo efficacy of a paclitaxel bifunctional against a human xenograft tumor cell line MDA-MB-435 in mice. The data show that the drug activity is maintained in the presence of the bifunctional modification.

### Examples 6

### Optimizing Extra and Intra-Cellular Distribution

The choice of pharmacokinetic modulating moiety is used to bias extra and intracellular distribution. The bias is dependent on the choice of drug target. Drugs such as insulin operate on extracellular receptors and there is no efficacy advantage to internalizing the protein to the intracellular space. However, many chemotherapeutics such as paclitaxel bind to an intracellular component such as tubulin, thus making an intracellular bias desirable. Nonetheless, overly biasing the distribution in the intracellular case will make it impossible for the drug to spread its effect over a large number of cells given the limited dose amount (typically 130 mg/kg in humans). Overbiasing the drug in the extracellular case will make it difficult to target the drug to specific locations if the therapeutic must cross cell membranes to achieve effective transport.

So, the tuning of the extracellular and intracellular distribution is important, and must be engineered using both the K_{D} and k_{off} parameters of the bifunctional binding to the drug target and bifunctional binding to the recruited biomoiety. To target blood cells intracellularly, it is desirable to target specific intracellular proteins such as FKBP-12, Erp-1, or Es-1. Ligands must also be optimized to cross the cell membrane. Analysis of the selected pool of ligands may proceed by the analysis below.

Once a decision is made for either an extra- or intra-cellular bias, the ligand is chosen accordingly. Moreover the ligand is designed to strike the correct balance to allow cell membrane transport where necessary. The bias may determined kinetically as described above by determining affinity and kinetic parameters for the bifunctional with respect to the drug target and recruited biomoiety. Kinetic and endpoint distributions in plasma and whole blood are determined by liquid chromatography and mass spectroscopy.

### Example 7

### Synthesis of Warfarin-SLF Bifunctional Compound

A warfarin-SLF bifunctional compound was synthesized as indicated in FIG. 12. The starting material is acenocoumarin (250 mg purchased from Toronto Research Chemicals, North York, Ontario, Canada). The NO₂ group on the acenocoumarin provides a convenient attachment point for synthesizing the bifunctional.

### Example 8

### Time Course of Warfarin-SLF Anticoagulant Effect

The time course of the anticoagulant effect of a warfarin-SLF bifunctional molecule, warfarin, and a vehicle control were measured. The results are depicted in FIG. 13.

Five mice per arm (fifteen animals total) were used for the test. The warfarin-SLF bifunctional molecule was that described in Example 7. The dose was 1.5 mg/kg, administered in 0.5% methylcellulose by gavage. The Helena Thromboplastic Reagent MI test (Helena Laboratories, Beaumont, Tex.) was employed to determine the duration of clotting.

It may be observed in the figure that the clotting time doubles after only about 8 hours for warfarin-SLF dosed mice as compared to 18 hours for warfarin-dosed mice. The faster onset of action can be explained because an intracellular depot for warfarin-SLF forms, allowing more rapid inhibition of vitamin K epoxide reductase. Warfarin is over 97% albumin bound and can exert its anticoagulant effect only by diffusing off the albumin and inside cells. Hence, the SLF moiety can alter the kinetics of drug onset.

1. A method for improving at least one pharmacokinetic property and efficacy of an anticancer therapeutic upon administration to a host, the method comprising:
administering to the host an effective amount of a bifunctional compound of less than about 5000 Daltons comprising the anticancer therapeutic or an active derivative, fragment or analog thereof and a pharmacokinetic modulating moiety,
wherein the pharmacokinetic modulating moiety binds to at least one intracellular protein,
wherein the bifunctional compound has at least one modulated pharmacokinetic property upon administration to the host and improved efficacy as compared to a free drug control that comprises the anticancer therapeutic, and
wherein the bifunctional compound gains in efficacy by intracellular sequestration.

2. The method according to claim 1, wherein the pharmacokinetic property is selected from the group consisting of half-life, hepatic first-pass metabolism, volume of distribution, and degree of blood protein binding.

3. The method according to claim 1, wherein the bifunctional compound is administered as a pharmaceutical preparation.

4. The method according to claim 1, wherein the host is a mammal.

5. The method according to claim 1 where the pharmacokinetic modulating moiety has a mass of less than 1100 Daltons and binds to a peptidyl prolyl isomerase.

6. The method according to claim 1, wherein the anti-cancer therapeutic targets VEG-F, bFGF, or PDGF, FGF2, or HGF.

7. The method according to claim 1, wherein the partitioning of the bifunctional compound between the extracellular and intracellular space improves pharmacokinetics.

8. The method according to claim 7, wherein the anticancer agent is bortezomib, bevacizumab, vendetanib, sunitinib, sorafenib, thalidomide, erlonitib, pegaptanib, or lenalidomide.

9. The method according to claim 1, wherein the pharmacokinetic modulating moiety binds to a cell surface receptor and the anticancer agent targets an angiogenic protein.

10. The method of claim 1 where the linker length is greater than or equal to 3 single bonded carbon atoms when measured via the shortest through-bond distance between drug moiety and linker moiety.

11. The method of claim 1 where the linker length is greater than or equal to 5 single bonded carbon atoms when measured via the shortest through-bond distance between drug moiety and linker moiety.

12. The method of claim 1 where the linker length is greater than or equal to 7 single bonded carbon atoms when measured via the shortest through-bond distance between drug moiety and linker moiety.

13. The method of claim 1 where the linker length is greater than or equal to 9 single bonded carbon atoms when measured via the shortest through-bond distance between drug moiety and linker moiety.

14. The method of claim 1 where the PMM is a ligand for an Es-1 erythrocyte receptor.

15. A method of synthesizing a bifunctional compound comprising warfarin and a synthetic ligand for FKBP, comprising (a) protecting the OH group in acenocoumarin with a protective moiety, (b) reducing the NO₂ group of acenocoumarin to NH₂, (c) coupling the NH₂ to a carboxylic acid moiety on the synthetic ligand thus forming a peptide bond, and (d) removing the protective moiety.

16. A bifunctional compound comprising (a) a coumarin-type anticoagulant or an active derivative, fragment, or analog thereof, (b) an optional linker, and (c) a synthetic ligand for FKBP, wherein oral administration of the bifunctional to mice results in a blood clotting time at 8 hours after oral administration which is at least about 50% greater than the blood clotting time resulting from oral administration of the same weight of the coumarin-type anticoagulant.

17. The bifunctional compound of claim 16, wherein the coumarin-type anticoagulant is chosen from the group consisting of warfarin, acenocoumarol, phenprocoumon, or dicumarol.

18. The bifunctional compound of claim 16, wherein oral bioavailability is not hindered compared to the coumarin-type anticoagulant by the presence of the synthetic ligand.
